(19) United States
(12) Patent Application Publication
(10) Pub. No.: US 2025/0241753 A1
(43) Pub. Date: Jul. 31, 2025

(54) Balloon Expandable Transcatheter Valve Delivery System

(71) Applicant: St. Jude Medical, Cardiology Division, Inc., St. Paul, MN (US)

(72) Inventors: Michael Shane Morrissey, St. Paul, MN (US); Robert Schulzetenberg, Columbia Heights, MN (US); Bradley Charles Knippel, Lino Lakes, MN (US); Corey Daniel Brown, Andover, MN (US); Parker Blezek, Minneapolis, MN (US); David Copeland, Minnetonka, MN (US); Tyler Govek, St. Louis Park, MN (US)

(73) Assignee: St. Jude Medical, Cardiology Division, Inc., St. Paul, MN (US)

(21) Appl. No.: 18/946,056

(22) Filed: Nov. 13, 2024

Related U.S. Application Data
(60) Provisional application No. 63/627,413, filed on Jan. 31, 2024.

(51) Int. Cl. A61F 2/24; A61F 2/95; A61M 25/01

(52) U.S. Cl. CPC A61F 2/2433; A61F 2/2418; A61F 2/9517; A61F 2230/0069; A61M 25/0136

(57) ABSTRACT

A prosthetic heart valve delivery system may include a handle, a delivery catheter with an inflatable balloon at a distal end thereof, and a prosthetic heart valve. An axial adjustment actuator may be positioned on the handle for translating the prosthetic heart valve distally or proximally relative to the handle when the prosthetic heart valve is collapsed onto the balloon. A commissure alignment actuator may be positioned on the handle for rotating the prosthetic heart valve about its central longitudinal axis when the prosthetic heart valve is collapsed onto the balloon. The axial adjustment and the commissure alignment actuators may be independently actuated, so that actuation of the axial adjustment actuator does not rotate the prosthetic heart valve, and actuation of the commissure alignment actuator does not translate the prosthetic heart valve.

### CROSS-REFERENCE TO RELATED APPLICATIONS

This application claims priority to the filing date of U.S. Provisional Patent Application No. 63/627,413, filed Jan. 31, 2024, the disclosure of which is hereby incorporated by reference herein.

### BACKGROUND OF THE DISCLOSURE

Valvular heart disease, and specifically aortic and mitral valve disease, is a significant health issue in the United States. Valve replacement is one option for treating heart valve diseases. Prosthetic heart valves include surgical heart valves, as well as collapsible and expandable heart valves intended for transcatheter aortic valve replacement or implantation (“TAVR” or “TAVI”) or transcatheter mitral valve replacement (“TMVR”). Surgical or mechanical heart valves may be sutured into a native annulus of a patient during an open-heart surgical procedure, for example. Collapsible and expandable heart valves may be delivered into a patient via a delivery apparatus such as a catheter to avoid a more invasive procedure such as full open-chest, open-heart surgery. As used herein, reference to a “collapsible and expandable” heart valve includes heart valves that are formed with a small cross-section that enables them to be delivered into a patient through a catheter in a minimally invasive procedure, and then expanded to an operable state once in place, as well as heart valves that, after construction, are first collapsed to a small cross-section for delivery into a patient and then expanded to an operable size once in place in the valve annulus.

Collapsible and expandable prosthetic heart valves typically take the form of a one-way valve structure (often referred to as a valve assembly) mounted within an expandable frame (the terms “stent” and “frame” may be used interchangeably herein). In general, these collapsible and expandable heart valves include a self-expanding, mechanically-expandable, or balloon-expandable frame, often made of nitinol or another shape-memory metal or metal alloy (for self-expanding frames) or steel or cobalt chromium (for balloon-expandable frames). The one-way valve assembly mounted to/within the stent includes one or more leaflets and may also include a cuff or skirt. The cuff may be disposed on the stent's interior or luminal surface, its exterior or abluminal surface, and/or on both surfaces. A cuff helps to ensure that blood does not just flow around the valve leaflets if the valve or valve assembly is not optimally seated in a valve annulus. A cuff, or a portion of a cuff disposed on the exterior of the stent, can help prevent leakage around the outside of the valve (the latter known as paravalvular or “PV” leakage).

Balloon expandable valves are typically delivered to the native annulus while collapsed (or “crimped”) onto a deflated balloon of a balloon catheter, with the collapsed valve being either covered or uncovered by an overlying sheath. Once the crimped prosthetic heart valve is positioned within the annulus of the native heart valve that is being replaced, the balloon is inflated to force the balloon-expandable valve to transition from the collapsed or crimped condition into an expanded or deployed condition, with the prosthetic heart valve tending to remain in the shape into which it is expanded by the balloon. Typically, when the position of the collapsed prosthetic heart valve is determined to be in the desired position relative to the native annulus (e.g. via visualization under fluoroscopy), a fluid (typically a liquid although gas could be used as well) such as saline is pushed via a syringe (manually, automatically, or semi-automatically) through the balloon catheter to cause the balloon to begin to fill and expand, and thus cause the overlying prosthetic heart valve to expand into the native annulus.

### SUMMARY OF THE DISCLOSURE

According to a first aspect of the disclosure, a prosthetic heart valve delivery system includes a handle, a delivery catheter extending distally from the handle, an inflatable balloon positioned at a distal end portion of the delivery catheter, and a prosthetic heart valve configured to be collapsed onto the inflatable balloon for transcatheter delivery of the prosthetic heart valve to a native heart valve of a patient. An axial adjustment actuator may be positioned on the handle, and actuation of the axial adjustment actuator may cause the prosthetic heart valve to translate distally or proximally relative to the handle when the prosthetic heart valve is collapsed onto the inflatable balloon. A commissure alignment actuator may be positioned on the handle, and actuation of the commissure alignment actuator may cause the prosthetic heart valve to rotate about a central longitudinal axis of the prosthetic heart valve when the prosthetic heart valve is collapsed onto the inflatable balloon. The axial adjustment actuator and the commissure alignment actuator may be configured to be independently actuated so that actuation of the axial adjustment actuator does not cause the prosthetic heart valve to rotate about the central longitudinal axis of the prosthetic heart valve, and actuation of the commissure alignment actuator does not cause the prosthetic heart valve to translate distally or proximally relative to the handle. The delivery catheter may include an outer catheter, and an inner catheter radially inward of the outer catheter. A catheter hub may be positioned at least partially within the handle, and the catheter hub may be axially and rotatably fixed to the inner catheter. An axial adjustment carrier may be mounted to the catheter hub so that the axial adjustment carrier is axially fixed relative to the catheter hub but is rotatable about the catheter hub. The axial adjustment carrier may include an extension, and the axial adjustment actuator may include internal threading, the extension engaging the internal threading. The extension may extend through a slot formed in the handle, the slot defining a maximum length of translation of the axial adjustment carrier relative to the handle. The catheter hub may include a keyed portion received within a keyed recess of the commissure alignment actuator so that rotation of the commissure alignment actuator causes corresponding rotation of the catheter hub.

The keyed portion may be hexagonal and the keyed recess may be hexagonal. The keyed portion may be positioned at a proximal end portion of the catheter hub, and the keyed portion may have a length, the length being selected so that the keyed portion remains engaged with the keyed recess in all achievable axial positions of the catheter hub relative to the handle. A hub housing may at least partially surround the catheter hub, the hub housing including an inflation port configured to receive a first end of an inflation tubing. The hub housing may be axially and rotationally fixed relative to the housing. A first seal may be engaged to an outer surface of the catheter hub at a first location, and a second seal may be engaged to the outer surface of the catheter hub at a second location, the first seal and the second seal also engaged to an inner surface of the hub housing. The catheter hub may include at least one aperture in a wall of the catheter hub at a location between the first seal and the second seal to create a first fluid pathway from (a) the inflation port into a void space defined between (i) the inner surface of the hub housing, (ii) the outer surface of the catheter hub, (iii) the first seal, and (iv) the second seal, and (b) from the void space into an interior lumen of the catheter hub. The inner catheter may be fixed to create a second fluid pathway from the interior lumen of the catheter hub, through the inner catheter, and to the balloon, the first fluid pathway being continuous with the second fluid pathway A pressure sensor may be in contact with the first fluid pathway and/or the second fluid pathway. The catheter hub may be rotatable and translatable relative to the hub housing. The catheter hub may have a total range of translation relative to the hub housing, and at all points within the total range of translation, the inflation port is positioned between the first seal and the second seal.

A steering actuator may be coupled to the handle, and a carriage within the handle may be engaged with the steering actuator so that actuation of the steering actuator causes distal or proximal translation of the carriage relative to the handle. A steering wire may have a proximal end portion coupled to the carriage, and a distal end portion coupled to a steering ring on a distal end portion of the delivery catheter, so that distal or proximal translation of the carriage causes deflection of the distal end portion of the delivery catheter. A deflection indicator slot may be formed in the handle, and a deflection indicator may be coupled to the carriage and extend through the deflection indicator slot so that a proximal-to-distal position of the deflection indicator relative to the deflection indicator slot indicates an amount of deflection of the distal end portion of the delivery catheter.

### BRIEF DESCRIPTION OF THE DRAWINGS

FIG. 1 is a perspective view of an example of a prosthetic heart valve.

FIG. 2 is a front view of an example of a section of the frame of the prosthetic heart valve of FIG. 1, as if cut longitudinally and laid flat on a table.

FIG. 3 is a front view of an example of a prosthetic leaflet of the prosthetic heart valve of FIG. 1, as if laid flat on a table.

FIG. 4 is a top view of the prosthetic heart valve of FIG. 1 mounted on an example of a portion of a delivery system.

FIG. 5 is an enlarged view of the handle of the delivery system shown in FIG. 4.

FIG. 6 is an enlarged view of a distal end of the delivery system shown in FIG. 4.

FIG. 7 is a top view of an example of a balloon catheter when the balloon is inflated.

FIG. 8 is a top view of an example of an inflation system for use with a delivery system similar to that shown in FIG. 4.

FIG. 9 is a side view of the inflation system of FIG. 8.

FIG. 10 is a perspective view of a connection between the inflation system of FIGS. 8-9 and the handle of the delivery system of FIG. 4.

FIG. 11 is a flowchart showing exemplary steps in a procedure to implant the prosthetic heart valve of FIG. 1 into a patient using the delivery system of FIG. 4.

FIG. 12 is a top view of an example of a handle of a delivery system.

FIG. 13 is a top view of the delivery system of FIG. 12 shown with a delivery catheter thereof in a deflected state.

FIG. 14 is a perspective view of a distal end portion of the handle of FIG. 12 with a portion of the casing thereof removed.

FIG. 15 is a cross-section of a proximal end portion of the handle of FIG. 12.

FIG. 16 is a cutaway view of the proximal end portion of the handle of FIG. 12 with a portion of the casing thereof removed.

FIG. 17 is an end view of a proximal end of the handle of FIG. 12.

FIG. 18 shows the view of FIG. 15 with indicators of fluid pathways provided thereon.

FIG. 19 is a cross-section of the proximal end portion of an alternate version of the handle of FIG. 12.

FIG. 20 is an exploded view of a portion of the handle of FIG. 12.

FIG. 21 is a perspective view of a steering wire coupler.

FIG. 22 is a cross-section of a receiver for receiving the steering wire coupler.

FIG. 23 is a perspective view of a cap assembled to the receiver of FIGS. 20 and 22.

### DETAILED DESCRIPTION OF THE DISCLOSURE

As used herein, the term “inflow end” when used in connection with a prosthetic heart valve refers to the end of the prosthetic valve into which blood first enters when the prosthetic valve is implanted in an intended position and orientation, while the term “outflow end” refers to the end of the prosthetic valve where blood exits when the prosthetic valve is implanted in the intended position and orientation. Thus, for a prosthetic aortic valve, the inflow end is the end nearer the left ventricle while the outflow end is the end nearer the aorta. The intended position and orientation are used for the convenience of describing valves disclosed herein. However, it should be noted that the use of the valve is not limited to the intended position and orientation but may be deployed in any type of lumen or passageway. For example, although prosthetic heart valves are described herein as prosthetic aortic valves, those same or similar structures and features can be employed in other heart valves, such as the pulmonary valve, the mitral valve, or the tricuspid valve. Further, the term “proximal,” when used in connection with a delivery device or system, refers to a position relatively close to the user of that device or system when it is being used as intended, while the term “distal” refers to a position relatively far from the user of the device. In other words, the leading end of a delivery device or system is positioned distal to the trailing end of the delivery device or system, when the delivery device is being used as intended. As used herein, the terms “substantially,” “generally,” “approximately,” and “about” are intended to mean that slight deviations from absolute are included within the scope of the term so modified. As used herein, the prosthetic heart valves may assume an “expanded state” and a “collapsed state,” which refer to the relative radial size of the stent.

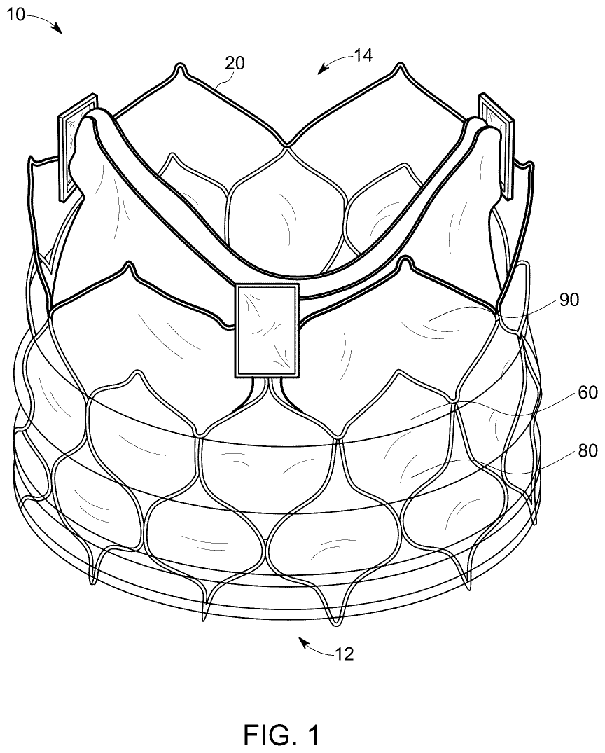

FIG. 1 is a perspective view of one example of a prosthetic heart valve 10. Prosthetic heart valve 10 may be a balloon-expandable prosthetic aortic valve, although in other examples it may be a self-expandable or mechanically-expandable prosthetic heart valve, intended for replacing a native aortic valve or another native heart valve. Prosthetic heart valve 10 is shown in an expanded condition in FIG. 1. Prosthetic heart valve 10 may extend between an inflow end 12 and an outflow end 14. Prosthetic heart valve 10 may include a collapsible and expandable frame 20, an inner cuff or skirt 60, an outer cuff or skirt 80, and a plurality of prosthetic leaflets 90. As should be clear below, prosthetic heart valve 10 is merely one example of a prosthetic heart valve, and other examples of prosthetic heart valves may be suitable for use with the concepts described below.

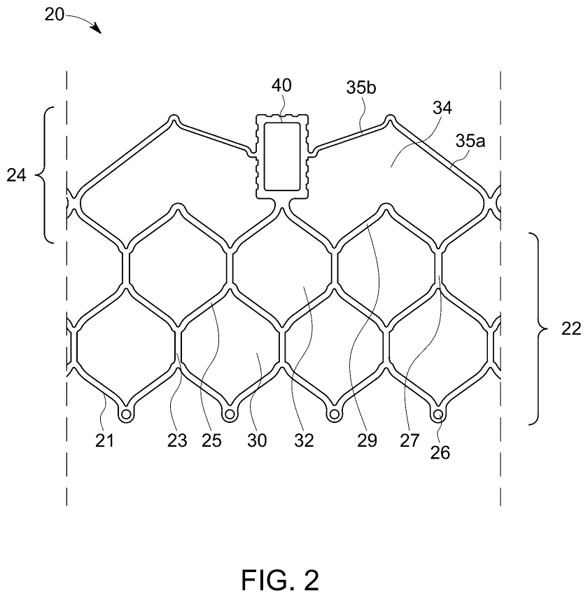

FIG. 2 is a front view of an example of a section of the frame 20 of prosthetic heart valve 10, as if cut longitudinally and laid flat on a table. The section of frame 20 in FIG. 2 may represent approximately one-third of a complete frame, particularly if frame 20 is used in conjunction with a three-leaflet prosthetic heart valve. In the illustrated example, frame 20 is a balloon-expandable stent and may be formed of stainless steel or cobalt-chromium, and which may include additional materials such as nickel and/or molybdenum. However, in some embodiments the stent may be formed of a shape memory material such as nitinol or the like. The frame 20, when provided as a balloon-expandable frame, is configured to collapse upon being crimped to a smaller diameter and/or expand upon being forced open, for example via a balloon within the frame expanding, and the frame will substantially maintain the shape to which it is modified when at rest.

Frame 20 may include an inflow section 22 and an outflow section 24. The inflow section 22 may also be referred to as the annulus section. In one example, the inflow section 22 includes a plurality of rows of generally hexagon-shaped cells. For example, the inflow section 22 may include an inflow-most row of hexagon-shaped cells 30 and an outflow-most row of hexagon-shaped cells 32. The inflow-most row of hexagonal cells 30 may be formed of a first circumferential row of angled or zig-zag struts 21, a second circumferential row of angled or zig-zag struts 25, and a plurality of axial struts 23 that connect the two rows. In other words, each inflow-most hexagonal cell 30 may be formed by two angled struts 21 that form an apex pointing in the inflow direction, two angled struts 25 that form an apex pointing in the outflow direction, and two axial struts that connect the two angled struts 21 to two corresponding angled struts 25. The outflow-most row of hexagonal cells 32 may be formed of the second circumferential row of angled or zig-zag struts 25, a third circumferential row of angled or zig-zag struts 29, and a plurality of axial struts 27 that connect the two rows. In other words, each outflow-most hexagonal cell 32 may be formed by two angled struts 25 that form an apex pointing in the inflow direction, two angled struts 29 that form an apex pointing in the outflow direction, and two axial struts that connect the two angled struts 27 to two corresponding angled struts 29. It should be understood that although the term “outflow-most” is used in connection with hexagonal cells 32, additional frame structure, described in more detail below, is still provided in the outflow direction relative to the outflow-most row of hexagonal cells 32.

In the illustrated embodiment, assuming that frame 20 is for use with a three-leaflet valve and thus the section shown in FIG. 2 represents about one-third of the frame 20, each row of cells 30, 32 includes twelve individual cells. However, it should be understood that more or fewer than twelve cells may be provided per row of cells. Further, the inflow or annulus section 22 may include more or fewer than two rows of cells. Still further, although cells 30, 32 are shown as being hexagonal, the some or all of the cells of the inflow section 22 may have other shapes, such as diamond-shaped, chevron-shaped, or other suitable shapes. In the illustrated embodiment, every cell 30 in the first row is structurally similar or identical to every other cell 30 in the first row, every cell 32 in the second row is structurally similar or identical to every other cell 32 in the second row, and every cell 30 in the first row is structurally similar or identical (excluding the aperture 26) to every cell 32 in the second row. However, in other examples, the cells in each row are not identical to every other cell in the same row or in other rows.

An inflow apex of each hexagonal cell 30 may include an aperture 26 formed therein, which may accept sutures or similar features which may help couple other elements, such as an inner cuff 60, outer cuff 80, and/or prosthetic leaflets 90, to the frame 20. However, in some examples, one or more or all of the apertures 26 may be omitted.

Still referring to FIG. 2, the outflow section 24 of the frame 20 may include larger cells 34 that have generally asymmetric shapes. For example, the lower or inflow part of the larger cells 34 may be defined by the two upper struts 29 of a cell 32, and one upper strut 29 of each of the two adjacent cells 32. In other words, the lower end of each larger cell 34 may be formed by a group of four consecutive upper struts 29 of three circumferentially adjacent cells 32. The tops of the larger cells 34 may each be defined by two linking struts 35a, 35b. The first linking strut 35a may couple to a top or outflow apex of a cell 32 and extend upwards at an angle toward a commissure attachment feature (“CAF”) 40. The second linking strut 35b may extend from an end of the first linking strut 35a back downwardly at an angle and connect directly to the CAF 40. To the extent that the larger cells 34 include sides, a first side is defined by a portion of the CAF 40, and a second side is defined by the connection between first linking strut 35a and the corresponding upper strut 29 of the cell 32 attached to the first linking strut 35a.

The CAF 40 may generally serve as an attachment site for leaflet commissures (e.g. where two prosthetic leaflets 90 join each other) to be coupled to the frame 20. In the illustrated example, the CAF 40 is generally rectangular and has a longer axial length than circumferential width. The CAF 40 may define an interior open rectangular space. The struts that form CAF 40 may be generally smooth on the surface defining the open rectangular space, but some or all of the struts may have one or more suture notches on the opposite surfaces. For example, in the illustrated example, CAF 40 includes two side struts (on the longer side of the rectangle) and one top (or outflow) strut that all include alternating projections and notches on their exterior facing surfaces. These projections and notches may help maintain the position of one or more sutures that wrap around these struts. These sutures may directly couple the prosthetic leaflets 90 to the frame 20, and/or may directly couple an intermediate sheet of material (e.g. fabric or tissue) to the CAF 40, with the prosthetic leaflets 90 being directly coupled to that intermediate sheet of material. In some embodiments, tabs or ends of the prosthetic leaflets 90 may be pulled through the opening of the CAF 40, but in other embodiments the prosthetic leaflets 90 may remain mostly or entirely within the inner diameter of the frame 20. It should be understood that balloon-expandable frames are typically formed of metal or metal alloys that are very stiff, particularly in comparison to self-expanding frames. At least in part because of this stiffness, although the prosthetic leaflets 90 may be sutured or otherwise directly coupled to the frame at the CAFs 40, it may be preferable that most or all of the remaining portions of the prosthetic leaflets 90 are not attached directly to the frame 20, but are rather attached directly to an inner skirt 60, which in turn is directly connected to the frame 20. Further, it should be understood that other shapes and configurations of CAFs 40 may be appropriate. For example, various other suitable configurations of frames and CAFs are described in greater detail in U.S. Provisional Patent Application No. 63/579,378, filed Aug. 29, 2023 and titled “TAVI Deployment Accuracy-Stent Frame Improvements,” the disclosure of which is hereby incorporated by reference herein.

With the example described above, frame 20 includes two rows of hexagon-shaped cells 30, 32, and a single row of larger cells 34. In a three-leaflet embodiment of a prosthetic heart valve that incorporates frame 20, each row of hexagon-shaped cells 30, 32 includes twelve cells, while the row of larger cells includes six larger cells 34. As should be understood, the area defined by each individual cell 30, 32 is significantly smaller than the area defined by each larger cell 34 when the frame 20 is expanded. There is also significantly more structure (e.g. struts) that create each row of individual cells 30, 32 than structure that creates the row of larger cells 34.

One consequence of the above-described configuration is that the inflow section 22 has a higher cell density than the outflow section 24. In other words, the total numbers of cells, as well as the number of cells per row of cells, is greater in the inflow section 22 compared to the outflow section 24. The configuration of frame 20 described above may also result in the inflow section 22 being generally stiffer than the outflow section 24 and/or more radial force being required to expand the inflow section 22 compared to the outflow section 24, despite the fact that the frame 20 may be formed of the same metal or metal alloy throughout. This increased rigidity or stiffness of the inflow section 22 may assist with anchoring the frame 20, for example after balloon expansion, into the native heart valve annulus. The larger cells 34 in the outflow section 24 may assist in providing clearance to the coronary arteries after implantation of the prosthetic heart valve 10. For example, after implantation, one or more coronary ostia may be positioned above the frame 20, for example above the valley where two adjacent larger cells 34 meet (about halfway between a pair of circumferentially adjacent CAFs 40). Otherwise, one or more coronary ostia may be positioned in alignment with part of the large interior area of a larger cell 34 after implantation. Either way, blood flow to the coronary arteries is not obstructed, and a further procedure that utilizes the coronary arteries (e.g. coronary artery stenting) will not be obstructed by material of the frame 20. Still further, the lower rigidity of the frame 20 in the outflow section 24 may cause the outflow section 24 to preferentially foreshorten during expansion, with the inflow section 22 undergoing a relatively smaller amount of axial foreshortening. This may be desirable because, as the prosthetic heart valve 10 expands, the position of the inflow end of the frame 20 may remain substantially constant relative to the native valve annulus, which may make the deployment of the prosthetic heart valve 10 more precise. This may be, for example, because the inflow end of the frame 20 is typically used to gauge proper alignment with the native valve annulus prior to deployment, so axial movement of the inflow end of the frame 20 relative to the native valve annulus during deployment may make precise placement more difficult.

Referring back to FIG. 1, the prosthetic heart valve 10 may include an inner skirt 60 mounted to the interior surface of frame 20. The inner skirt 60 may be formed of tissue, such as pericardium, although other types of tissue may be suitable. In the illustrated example, the inner skirt 60 is formed of a woven synthetic fabric, such as polyethylene terephthalate (“PET”) or polytetrafluoroethylene (“PTFE”), although other fabrics may be suitable, including fabrics other than woven fabrics. In some examples, the inner skirt 60 has straight or zig-zag shaped inflow and outflow ends that generally follow the contours of the cells 30, 32 of the inflow section 22 of frame 20. Preferably, inner skirt 60 is sutured to the frame 20 along the struts that form cells 30, 32. If apertures 26 are included, inner skirt 60 may also be coupled to frame 20 via sutures passing through apertures 26. Preferably, the inner skirt 60 does not cover (or does not cover significant portions of) the larger cells 34. The inner skirt 60 may be coupled to the frame 20 via mechanisms other than sutures, including for example ultrasonic welding or adhesives. Further, the inner skirt 60 may have shapes other than that shown, and need not have a zig-zag inflow or outflow end, and need not cover every cell in the inflow section 22. In fact, in some examples, the inner skirt 60 may be omitted entirely, with the outer skirt 80 (described in greater detail below) being the only skirt used with prosthetic heart valve 10. If the inner skirt 60 is provided, it may assist with sealing the prosthetic heart valve 10 within the heart, as well as serving as a mounting structure for the prosthetic leaflets 90 (described in greater detail below) within the frame 20.

Still referring to FIG. 1, the prosthetic heart valve 10 may include an outer skirt 60 mounted to the exterior surface of frame 20. The outer skirt 80 may be formed of tissue, such as pericardium, although other types of tissue may be suitable. In the illustrated example, the outer skirt 80 is formed of a woven synthetic fabric, such as PET or PTFE, although other fabrics may be suitable, including fabrics other than woven fabrics. In some examples, the outer skirt 80 has straight or zig-zag inflow end. Preferably, outer skirt 80 is sutured to the frame 20 and/or inner skirt 60 along the inflow edge of the outer skirt 80. If apertures 26 are included, outer skirt 80 may also be coupled to frame 20 via sutures passing through apertures 26. The outer skirt 80 may include a plurality of folds or pleats, such a circumferentially extending folds or pleats. The folds or pleats may be formed in the outer skirt 80 via heat setting, for example by placing the outer skirt 80 within a mold that forces the outer skirt 80 to form folds of pleats, and the outer skirt 80 may be treated with heat so that the outer skirt 80 tends to maintain folds or pleats in the absence of applied forces. The outflow edge of outer skirt 80 may be coupled to the frame 20 at selected, spaced apart locations around the circumference of the frame 20. In some embodiments, the outflow edge of outer skirt 80 may be connected to the inner skirt 60 along a substantially continuous suture line. Some or all of the outer skirt 80 between its inflow and outflow edges may remain not directly couples to the frame 20 or inner skirt 60. Preferably, the outer skirt 80 does not cover (or does not cover significant portions of) the larger cells 34. In use, the outer skirt 80 may directly contact the interior surface of the native heart valve annulus to assist with sealing, including sealing against PV leak. If folds or pleats are included with the outer skirt 80, the additional material of the folds or pleats may help further mitigate PV leak. However, it should be understood that the folds or pleats may be omitted from outer skirt 80, and the outer skirt 80 may have shapes other than that shown. In fact, in some examples, the outer skirt 80 may be omitted entirely, with the inner skirt 60 being the only skirt used with prosthetic heart valve 10. If the inner skirt 60 is omitted, the prosthetic leaflets 90 may be attached directly to the frame 20 and/or directly to the outer skirt 80.

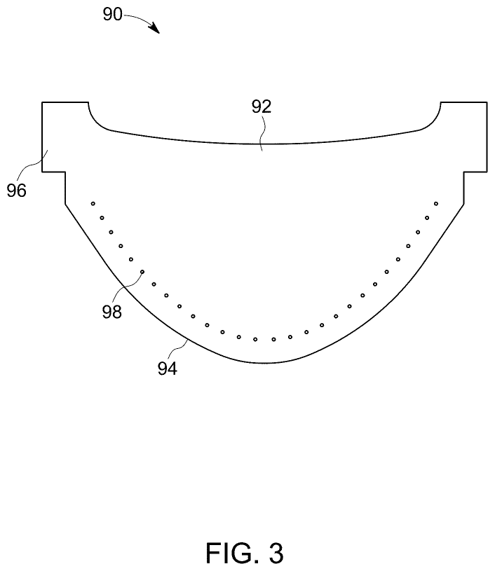

FIG. 3 is a front view of a prosthetic leaflet 90, as if laid flat on a table. In the illustrated example of prosthetic heart valve 10, a total of three prosthetic leaflets 90 are provided, although it should be understood that more or fewer than three prosthetic leaflets may be provided in other example of prosthetic heart valves. The prosthetic leaflet 90 may be formed of a synthetic material, such a polymer sheet or woven fabric, or a biological material, such a bovine or porcine pericardial tissue. However, other materials may be suitable. In on example, the prosthetic leaflet 90 is formed to have a concave free edge 92 configured to coapt with the free edges of the other leaflets to help provide the one-way valve functionality. The prosthetic leaflet 90 may include an attached edge 94 which is attached (e.g. via suturing) to other structures of the prosthetic heart valve 10. For example, the attached edge 94 may be coupled directly to the inner skirt 60, directly to the frame 20, and/or directly to the outer skirt 80. It may be preferable that the attached edge 94 is coupled directly only to the inner skirt 60, which may help reduce stresses on the prosthetic leaflet 90 compared to if the attached edge 94 were coupled directly to the frame 20. In some embodiments, a plurality of holes 98 may be formed along the attached edge 94 (or a spaced distance therefrom), for example via lasers. If included, the holes 98 may be used to receive sutures therethrough, which may make it easier to couple the prosthetic leaflet 90 to the inner skirt 60 during manufacturing. For example, the holes 98 may serve as guides if suturing is performed manually, and if the positions of the holes 98 are controlled via the use of layers, the holes 98 may be consistently placed among different prosthetic leaflets 90 to reduce variability between different prosthetic leaflets 90. Laflet tabs 96 may be provided at the junctions between the free edge 92 and the attached edge 94. Each leaflet tab 96 may be joined to a leaflet tab of an adjacent prosthetic leaflet to form prosthetic leaflet commissures, which may be coupled to the frame 20 via CAFs 40.

The prosthetic heart valve 10 may be delivered via any suitable transvascular route, for example transapically or transfemorally. Generally, transapical delivery utilizes a relatively stiff catheter that pierces the apex of the left ventricle through the chest of the patient, inflicting a relatively higher degree of trauma compared to transfemoral delivery. In a transfemoral delivery, a delivery device housing or supporting the valve is inserted through the femoral artery and advanced against the flow of blood to the left ventricle. In either method of delivery, the valve may first be collapsed over an expandable balloon while the expandable balloon is deflated. The balloon may be coupled to or disposed within a delivery system, which may transport the valve through the body and heart to reach the aortic valve, with the valve being disposed over the balloon (and, in some circumstances, under an overlying sheath). Upon arrival at or adjacent to the aortic valve, a surgeon or operator of the delivery system may align the prosthetic valve as desired within the native valve annulus while the prosthetic valve is collapsed over the balloon. When the desired alignment is achieved, the overlying sheath, if included, may be withdrawn (or advanced) to uncover the prosthetic valve, and the balloon may then be expanded causing the prosthetic valve to expand in the radial direction, with at least a portion of the prosthetic valve foreshortening in the axial direction.

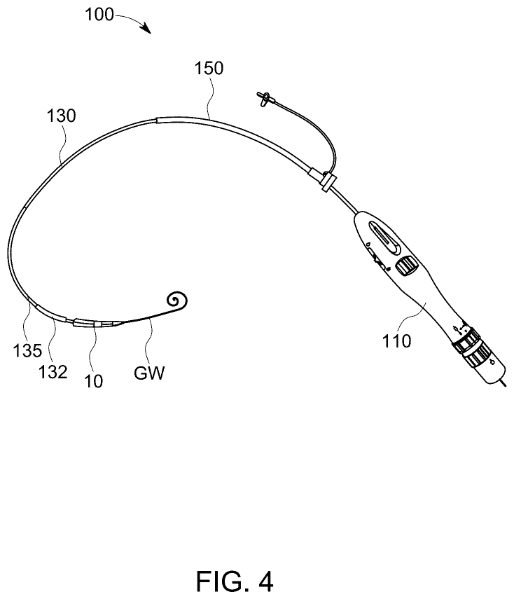

FIG. 4 illustrates one example of a delivery system 100, with the prosthetic heart valve 10 crimped over a balloon on a distal end of the delivery system 100. Although delivery system 100 and various components thereof are described below, it should be understood that delivery system 100 is merely one example of a balloon catheter that may be appropriate for use in delivering and deploying prosthetic heart valve 10.

In some examples, delivery system 100 includes a handle 110 and a delivery catheter 130 extending distally from the handle 110. An introducer 150 may be provided with the delivery system 100. Introducer 150 may be an integrated or captive introducer, although in other embodiments introducer 150 may be a non-integrated or non-captive introducer. In some examples, the introducer 150 may be an expandable introducer, including for example an introducer that expands locally as a large diameter components passes through the introducer, with the introducer returning to a smaller diameter once the large diameter components passes through the introducer. In other examples, the introducer 150 is a non-expandable introducer.

A guidewire GW may be provided that extends through the interior of all components of the delivery system 100, from the proximal end of the handle 110 through the atraumatic distal tip 138 of the delivery catheter 130. The guidewire GW may be introduced into the patient to the desired location, and the delivery system 100 may be introduced over the guidewire GW to help guide the delivery catheter 130 through the patient's vasculature over the guidewire GW.

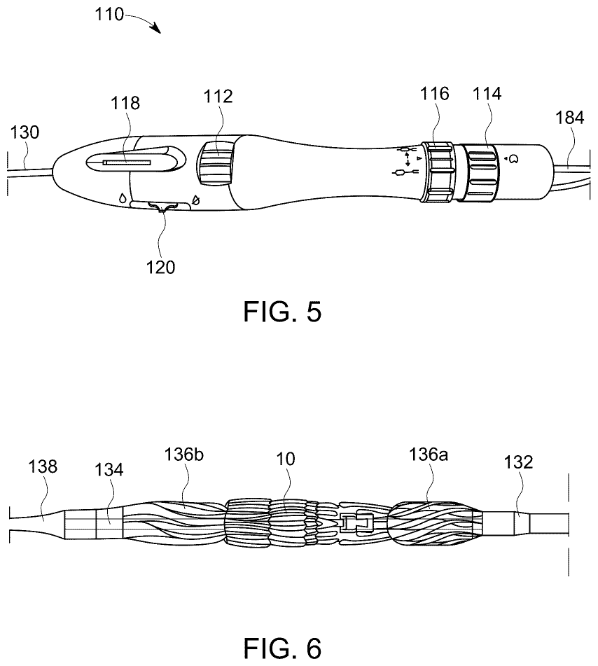

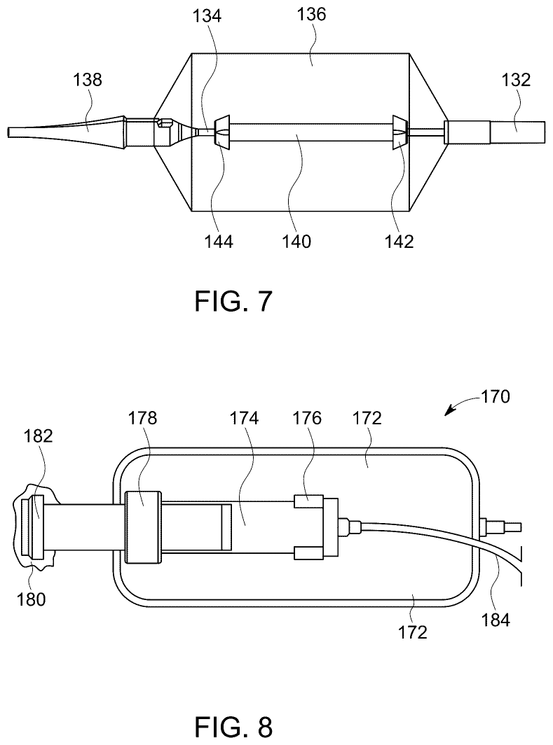

In some examples, the delivery catheter 130 is steerable. For example, one or more steering wires may extend through a wall of the delivery catheter 130, with one end of the steering wire coupled to a steering ring coupled to the delivery catheter 130, and another end of the steering wire operable coupled to a steering actuator on the handle 110. In such examples, as the steering actuator is actuated, the steering wire is tensioned or relaxed to cause deflection or straightening of the delivery catheter 130 to assist with steering the delivery catheter 130 to the desired position within the patient. For example, FIG. 5 is an enlarged view of the handle 110. Handle 110 may include a steering knob 112 that, upon rotation, tensions or relaxes the steering wires to deflect the distal end of the delivery catheter 130. However, it should be understood that the steering functionality may be omitted in some examples, and in other examples steering actuators other than knobs may be utilized. Further, in some examples, including those shown in FIGS. 6-7, the delivery catheter 130 includes an outer catheter 132, and an inner catheter 134. The inner catheter 134 may also be referred to as a guidewire catheter. The steering functionality may be provided in either the outer catheter 132, or the inner catheter 134, or in both catheters. However, in some examples, a separate steering catheter 135 may be provided. For example, as shown in FIG. 4, the steering catheter 135 may be positioned outside of the outer catheter 132 and may terminate just proximal to the balloon 136. With this configuration, deflection of the steering catheter 135 will also cause deflection of the outer catheter 132 and the inner catheter 134 which are both nested within the steering catheter 135. In some examples, the handle may include a window 118 that allows viewing of an indicator that corresponds to the amount of catheter deflection. For example, a carrier to which the indicator is attached may be attached to the steering wire. In some examples, when there is minimum (or zero) tension on the steering wire, the indicator is at the far distal position within window 118, but as deflection is actuated, for example by drawing a carrier proximally (and tensioning the steering wire as the carrier draws proximally), the indicator will move proximally along window 118, giving the user a readily-apparent indication of the amount of deflection applied to the catheter at any given moment.

Still referring to FIGS. 4-5, the delivery system 100 may include additional functionality to assist with positioning the prosthetic heart valve 10. For example, in the illustrated example, handle 110 includes a commissure alignment actuator 114, which may be positioned near a proximal end of the handle or at any other desired location. In the illustrated example, the commissure alignment actuator 114 is in the form of a rotatable knob, although other forms may be suitable. The commissure alignment knob 114 may be rotationally coupled to a portion of the delivery catheter 130 supporting the prosthetic heart valve 10. For example, the commissure alignment actuator 114 may be rotationally coupled to an inner catheter 134 which supports the prosthetic heart valve 10 in the crimped condition. With this configuration, rotating the commissure alignment knob 114 may cause the inner catheter 134 to rotate about its longitudinal axis, and thus cause the prosthetic heart valve 10 to rotate about its longitudinal axis. If a commissure alignment actuator 114 is included, it may be used to help ensure that, upon deployment of the prosthetic heart valve 10 into the native valve annulus, the commissures of the prosthetic heart valve are in rotational alignment with respective ones of the native valve commissures (e.g. within +/−2.5 degrees of rotational alignment, within +/−5 degrees of rotational alignment, within +/−10 degrees of rotational alignment, within +/−15 degrees of rotational alignment, etc.). Although commissure alignment actuator 114 is shown in this example as a knob positioned at or near a proximal end of the handle 110, it should be understood that the actuator 114 may take forms other than a knob, may be positioned at other suitable locations, and may be omitted entirely if desired.

Still referring to FIGS. 4-5, the delivery system 100 may include even further functionality to assist with positioning the prosthetic heart valve 10. For example, in the illustrated example, handle 110 includes an axial alignment actuator 116, which may be positioned near a proximal end of the handle, including distal to the commissure alignment actuator 114, or at any other desired location. In the illustrated example, the axial alignment actuator 116 is in the form of a rotatable knob, although other forms may be suitable. The axial alignment knob 116 may be operably coupled to a portion of the delivery catheter 130 supporting the prosthetic heart valve 10. For example, the axial alignment actuator 116 may include internal threads that engage external threads (or another component, such as individual extensions, which may be cylindrical extensions that fit between internal threads of the actuator) of a carriage that is coupled to an inner catheter 134 which supports the prosthetic heart valve 10 in the crimped condition. In such an example, the carriage may be rotatably fixed to the handle 110. With this configuration, rotating the axial alignment knob 116 may cause the carriage to advance distally or retract proximally as the inner threads of the axial alignment knob 116 mesh with the external threads of the carriage, but the carriage is prevented from rotating. As the carriage advances distally or retracts proximally, the inner catheter 134 may correspondingly advance distally or retract proximally, and thus cause the prosthetic heart valve 10 to advanced distally or retract proximally. It should be understood that, if axial alignment actuator 116 is included, it may have a small total range of motion, including for example between about 2 mm and about 15 mm of range of motion, including about 7.5 mm range of motion. In other words, the rough or coarse axial alignment between the prosthetic heart valve 10 and native valve annulus may be achieved by physically advancing the entire delivery catheter 130 by pushing it through the vasculature while holding the handle 110. However, for fine and more controlled adjustment of the axial position of the prosthetic heart valve 10 relative to the native valve annulus, which may be performed just prior to or during deployment of the prosthetic heart valve 10, the axial alignment knob 116 may be used. If an axial alignment actuator 116 is included, it may be used to help ensure that, upon deployment of the prosthetic heart valve 10 into the native valve annulus, the inflow end of the of the prosthetic heart valve is in axial alignment with the inflow aspect of the native valve annulus (e.g. within +/−0.5 mm of axial alignment, within +/−1.0 mm of axial alignment, within +/−1.5 mm of axial alignment, within +/−2.0 mm of axial alignment, etc.). Although axial alignment actuator 116 is shown in this example as a knob positioned at or near a proximal end of the handle 110, it should be understood that the actuator 116 may take forms other than a knob, may be positioned at other suitable locations, and may be omitted entirely if desired.

In addition to steering and positioning actuators, delivery system 100 may include a balloon actuator 120. In the illustrated example, balloon actuator 120 is positioned on the handle 110 near a distal end thereof, and is provided in the form of a switch. Balloon actuator 120 may be actuated to cause inflation or deflation of a balloon 136 that is part of the delivery system 100. For example, referring briefly to FIGS. 6-7, the delivery system 100 may include a balloon 136 that overlies a distal end of inner catheter 134 and which receives the prosthetic heart valve 10 in a crimped condition thereon. In the example illustrated in FIG. 6, the balloon 136 includes a proximal pillowed portion 136a, a distal pillowed portion 136b, and a central portion over which the prosthetic heart valve 10 is crimped. The proximal pillow 136a and the distal pillow 136b may form shoulders on each side of the prosthetic heart valve 10, which may help ensure the prosthetic heart valve 10 does not move axially relative to the balloon 136 and/or inner catheter 134 during delivery. The shoulder formed by the distal pillow 136 may also help protect the inflow edge of the prosthetic heart valve 10 from contact with the anatomy during delivery. For example, during a transfemoral delivery, as the distal end of the delivery catheter 130 traverse the sharp bends of the aortic arch (or during initial introduction into the patient), there is a relatively high likelihood the inflow end of the prosthetic heart valve 10 (which is the leading edge during transfemoral delivery) will contact a vessel wall (or a components of an introduction system) causing dislodgment of the prosthetic heart valve 10 relative to the balloon 136. The distal pillow 136 may tend to have an equal or larger outer diameter than the inflow end of the prosthetic heart valve 10 (when the prosthetic heart valve 10 is crimped and the balloon 136 is deflated), which may help ensure the inflow edge of the prosthetic heart valve 10 does not inadvertently contact another structure during delivery. In some examples, the pillowed portions 136a, 136b may be formed via heat setting. Additional related features for use in similar balloon catheter delivery systems are described in greater detail in U.S. Provisional Patent Application No. 63/382,812, filed Nov. 8, 2022 and titled “Prosthetic Heart Valve Delivery and Trackability,” the disclosure of which is hereby incorporated by reference herein.

In order to deploy the prosthetic heart valve 10, the balloon 136 is inflated, for example by actuating the balloon actuator 120 to force fluid (such as saline, although other fluids, including liquids or gases, could be used) into the balloon 136 to cause it to expand, causing the prosthetic heart valve 10 to expand in the process. For example, the balloon actuator 120 may be pressed forward or distally to cause fluid to travel through an inflation lumen within delivery catheter 130 to inflate the balloon 136. In some embodiments, the balloon actuator 120 may take the form of a “momentary switch” in which pushing the balloon actuator 120 forward engages inflation, pulling the balloon actuator 120 proximally engages deflation, and releasing the balloon actuator 120 pauses inflation. This particular example of functionality may allow the physician to precisely control the amount of fluid dispensed while reducing the occurrence of over-or under-inflation, for example because the system automatically pauses inflation when the switch is released. The physical form factor of the balloon actuator 120 may be any suitable desired form factor, including for example a rocker switch, a push button, etc. In some embodiments a second balloon actuator or button may be provided, either on the balloon actuator 120 or elsewhere on the handle 110, with the second balloon actuator allowing for a change (e.g. increase or decrease) in the rate of inflation, for example to a pre-programmed faster or slower rate of inflation. FIG. 7 illustrates an example of the balloon 136 after being inflated, with the prosthetic heart valve 10 omitted from the figure for clarity. In the illustrated example, the balloon 136 may be formed to have a distal end that is fixed to a portion of an atraumatic distal tip 138. The distal tip 138 may be tapered to help the delivery catheter 130 move through the patient's vasculature more smoothly. A proximal end of the balloon 136 may be fixed to a distal end of outer catheter 132. The inflation lumen may be the space between the outer catheter 132 and the inner catheter 134, or in other embodiments may be provided in a wall of the inner catheter 134, or in any other location that fluidly connects the interior of the balloon 136 to a fluid source outside of the patient that is operable coupled to the delivery system 100.

Referring to FIG. 7, in some examples, a mounting shaft 140 may be provided on the inner catheter 134. A proximal stop 142 and/or a distal stop 144 may be provided, for example at opposite ends of the mounting shaft 140. If the mounting shaft 140 is included, it may provide a location on which the prosthetic heart valve 10 may be crimped. If the proximal stop 142 and/or distal stop 144 is provided, they may provide physical barriers to the prosthetic heart valve 10 moving axially relative to the balloon 136. In one example, the proximal stop 142 may taper from a larger distal diameter to a smaller proximal diameter, and the distal stop may taper from a larger proximal diameter to a smaller distal diameter. The spacing between the proximal stop 142 and the distal stop 144, if both are included, may be slightly larger than the length of the prosthetic heart valve 10 when it is crimped over mounting shaft 140. However, it should be understood that one or both of the stops 142, 144 may be omitted, and the mounting shaft 140 may also be omitted. If the mounting shaft 140 is included, it is preferably axially and rotationally fixed to the inner catheter 134 so that movement of the inner catheter 134 causes corresponding movement of the mounting member 140, and thus the prosthetic heart valve 10 when mounted thereon.

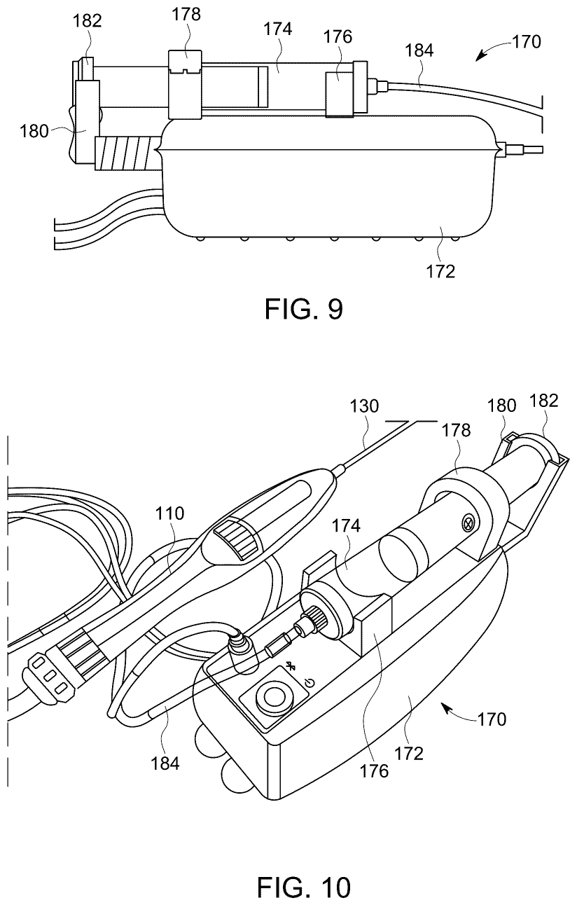

Before describing the use of balloon actuator 120 in more detail, it should be understood that in some embodiments, the balloon actuator 120 may be omitted and instead a manual device, such as a manual syringe, may be provided along with delivery system 100 in order to manually push fluid into balloon 136 during deployment of the prosthetic heart valve 10. However, in the illustrated example of delivery system 100, the balloon actuator 120 provides for a motorized and/or automated (or semi-automated) balloon inflation functionality. For example, FIG. 8 and FIG. 9 illustrate an example of a balloon inflation system 170. Balloon inflation system 170 may include a housing 172 that houses one or more components, which may include a motor, one or more batteries, electronics for control and/or communication with other components, etc. Housing 172 may include one or more fixed cradles to receive a syringe 174. In the illustrated embodiment, a distal cradle 176 is provide with an open “C”- or “U”-shaped configuration so that the distal end of the syringe 174 may be snapped into or out of the distal cradle 176. A proximal cradle 178 may also be provided, which may have a “C”- or “U”-shaped bottom portion hingedly connected to a “C”- or “U”-shaped top portion. This configuration may allow for the proximal end of the outer body of the syringe 174 to be snapped into the bottom portion of proximal cradle 178, and the top portion of proximal cradle 178 may be closed and connected to the bottom portion to fully circumscribe the outer body of the syringe 174 to lock the syringe 174 to the housing 172. It should be understood that more or fewer cradles, of similar or different designs, may be included with housing 172 to help secure the syringe 174 to the housing 172 in any suitable fashion.

The balloon inflation system 170 may include a moving member 180. In the illustrated embodiment, moving member 180 includes a “C”- or “U”-shaped cradle to receive a plunger handle 182 of the syringe 174 therein, the cradle being attached to a carriage that extends at least partially into the housing 172. The carriage of the moving member 180 may be generally cylindrical, and may include internal threading that mates with external threading of a screw mechanism (not shown) within the housing 172 that is operably coupled to a motor. In some embodiments, the carriage may have the general shape of a “U”-beam with the flat face oriented toward the top. The moving member 180 may be rotationally fixed to the housing 172 via any desirable mechanism, so that upon rotation of the screw mechanism by the motor, the moving member 180 advances farther into the housing 172, or retracts farther away from the housing 172, depending on the direction of rotation of the screw mechanism. While the plunger handle 182 is coupled to the moving member 180, advancement of the moving member 180 forces fluid from the syringe 174 toward the balloon 136, while retraction of the moving member 180 withdraws fluid from the balloon 136 toward the syringe 174. It should be understood that the motor, or other driving mechanism, may be located in or outside the housing 172, and any other suitable mechanism may be used to operably couple the motor or other driving mechanism to the moving member 180 to allow for axial driving of the plunger handle 182.

As shown in each of FIG. 8, FIG. 9, and FIG. 10, the distal end of syringe 174 may be coupled to tubing 184 that is in fluid communication with an inflation lumen of delivery catheter 130 that leads to the balloon 136 at or near the distal end of the delivery system 100. Tubing 184 may allow for the passage of the fluid (e.g., saline) from the syringe 174 toward the balloon 136, or for withdrawal of fluid from the balloon 136 toward the syringe 174, for example based on whether the balloon actuator 120 is pressed forward or backward.

Although not separately numbered in FIG. 8, FIG. 9, and FIG. 10, the housing 172 may include one or more cables extending from the housing, for example to allow for transmission of power (e.g. from AC mains or another component with which the cable is coupled) and/or transmission of data, information, control commands, etc. For example, one cable may couple the housing 172 to handle 110 so that controls on the handle 110 (e.g. balloon actuator 120) may be used to activate the balloon inflation system 170 in the desired fashion. Another cable may couple to a computer display or similar device to provide information regarding the inflation of the balloon 136. However, it should be understood that any transmission of data or information may be provided wirelessly instead of via a wired connection, for example via a Bluetooth or other suitable connection. Additional and related features of balloon inflation system 170, related systems, and the uses thereof are described in U.S. patent application Ser. No. 18/311,458, the disclosure of which is hereby incorporated by reference herein.

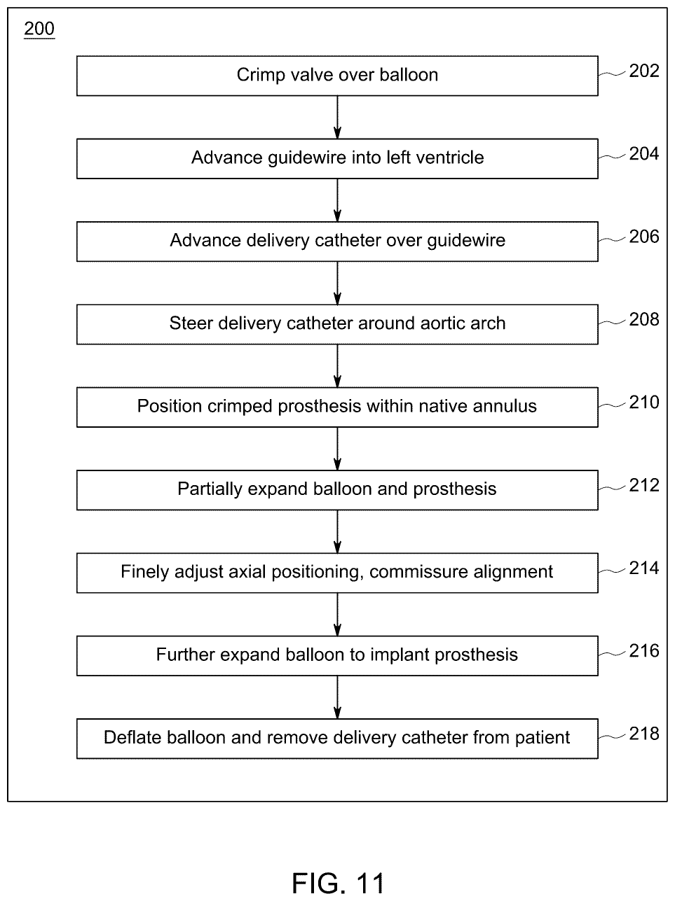

FIG. 11 is a flowchart showing exemplary steps in an implantation procedure 200 to implant the prosthetic heart valve 10 of FIG. 1 into a patient using the delivery system 100 of FIG. 4. However, it should be understood that not all of the steps shown in connection with implantation procedure 200 need to be performed, and various steps not explicitly shown and described in connection with procedure 200 may be performed as part of the implantation procedure. At the beginning of the procedure 200 in step 202, the prosthetic heart valve 10 may be collapsed over or crimped onto balloon 136, with the balloon 136 being mostly or entirely deflated after the crimping procedure. It should be understood that crimping step 202 may be performed at any time prior to the procedure, including at the beginning of the procedure, or at an earlier stage before the delivery system 100 is provided to the end user. In other words, the crimping step 202 may be performed during a manufacturing stage of the delivery system 100 and/or prosthetic heart valve 10. During an early stage of the implantation procedure 200, a guidewire GW may be advanced into the patient in step 204, for example via the femoral artery, around the aortic arch, through the native aortic valve, and into the left ventricle. The guidewire GW may be used as a rail for other devices that need to access this pathway. For example, in step 206, the atraumatic distal tip 138 may be advanced over the proximal end of the guidewire GW, and the delivery catheter 130 may be advanced over guidewire GW toward the native aortic valve. During this initial advancement of the delivery catheter 130 into the patient, the introducer 150 (if included) may be positioned distally, for example so that it covers the prosthetic heart valve 10 or so that it is positioned just proximal to the prosthetic heart valve 10. Advancement of the delivery catheter 130 and introducer 150 may continue until a proximal hub of the introducer is in contact with the patient's skin (or in contact with another device that enters the patient's femoral artery. At this point, the introducer 150 may stop moving axially relative to the patient, with the delivery catheter 130 continuing to advance relative to the introducer 150. If steering capability is provided, the delivery catheter 130 may be steered or deflected at any point to assist with achieving the desired pathway of the delivery catheter 130. As on example, in step 208, the steering knob 112 may be actuated to deflect the distal end of the delivery catheter 130 as it traverses the sharp bends of the aortic arch. Advancement of the delivery catheter 130 may continue in step 210 until the prosthetic heart valve 10, while still crimped or collapsed, is positioned within the native aortic valve annulus. With the desired position achieved, the balloon 136 may be partially inflated, for example by pressing balloon actuator 120 forward, to partially expand the prosthetic heart valve 10 in step 212. In some examples, it is desirable to expand the prosthetic heart valve 10 only partially in step 212, because the position of the prosthetic heart valve 10 (including rotational and/or axial positioning) relative to the native aortic valve annulus may shift during this partial expansion. After the partial expansion of step 212, the user may examine the positioning of the prosthetic heart valve 10 relative to the native aortic valve annulus. If desired, in step 214, the axial positioning of the partially-expanded prosthetic heart valve 10 relative to the native aortic valve annulus may be finely adjusted (e.g. by actuating axial alignment actuator 116) and/or the rotational orientation of the prosthetic heart valve 10 relative to the native aortic valve may be finely adjust (e.g. by actuating commissure alignment actuator 114). When the desired axial alignment is achieved and the desired rotational alignment (e.g. rotational alignment between the prosthetic commissure and the native commissures) is achieved, the balloon 136 may be fully expanded in step 216 to fully expand the prosthetic heart valve 10 and to anchor the prosthetic heart valve 10 in the native aortic valve annulus in the desired position and orientation. After deployment is complete, the balloon 136 may be deflated in step 218, for example by pressing actuating balloon 120 backward, and the delivery catheter 130 and guidewire GW may be removed from the patient to complete the procedure. It should be understood that the nine steps shown in FIG. 11 as part of procedure 200 are merely exemplary of a single example of an implantation procedure, and steps shown may be omitted, steps not shown may be included, and steps may be provided in any order deemed appropriate by the physician and/or medical personnel.

Although various components of a prosthetic heart valve 10 and delivery system 100 are described above, it should be understood that these components are merely intended to provide better context to the systems, features, and/or methods described below. Thus, various components of the systems described above may be modified or omitted as appropriate without affecting the systems, features, and/or methods described below. For example, prosthetic heart valves other than the specific configuration shown and described in connection with FIGS. 1-3 may be used with delivery systems other than the specific configuration shown and described in connection with FIGS. 4-10 as part of an implantation procedure that uses steps other than the specific configuration shown and described in connection with FIG. 11, without affecting the inventive systems, features, and/or methods described below.

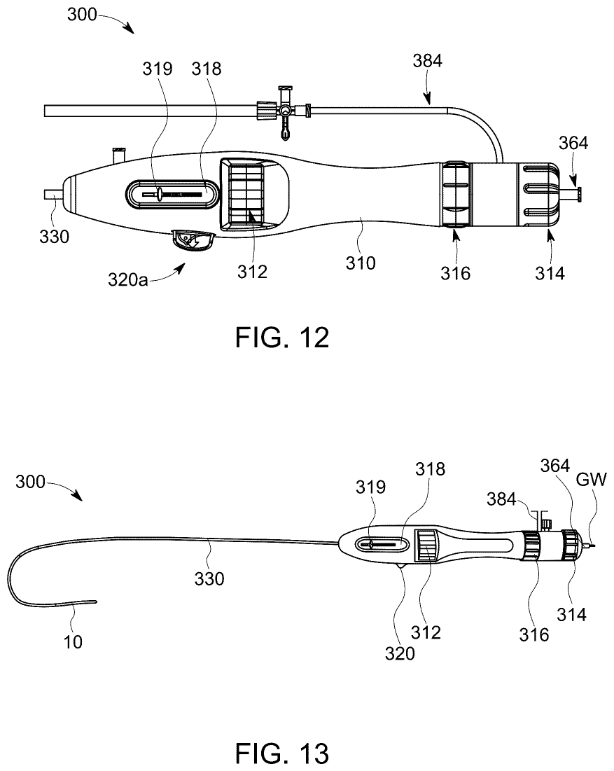

Although various functions of delivery system 100 are describe in greater detail above, such functions may be achieved in various ways and alternative features and/or structures may be provided. For example, a delivery system 300 is shown and described below in connection with FIGS. 12-19. These features generally include part numbers in the 300-series that correspond to similar or identical features of delivery system 100 provided with part numbers in the 100-series. It should be understood that features of delivery system 300 may be combined with or used in place of features of delivery system 100, and vice versa. For example, although an inflation system 170 is not specifically shown and described in detail in connection with delivery system 300, the inflation system 170 may be used with delivery system 300 with little or no modification.

FIG. 12 is a top view of a handle 310 of delivery system 300. Handle 310 may be similar to handle 110 in many aspects. For example, handle 310 may be formed with a two-part casing, although in some embodiments handle 310 may be formed from a one-part casing or more than two-parts casing. Handle 310 may be generally configured for delivery and deploying a prosthetic heart valve, particularly a balloon-expandable prosthetic heart valve, including but not limited to prosthetic heart valve 10. Extending from the distal end of handle 310 may be delivery catheter 330, which may be similar or identical to delivery catheter 130. In the view of FIG. 12, only a steering catheter 335 of the delivery catheter 330 is visible, although as should be understood from the below description of delivery system 300 (as well as the above description of delivery system 100), the delivery catheter 330 may include components (including portions of steering catheter 335, outer catheter 332 and inner catheter 334) that are housed within the handle 310.

As with delivery system 100, delivery system 300 may include one or more functions to assist with delivering and deploying a prosthetic heart valve (such as prosthetic heart valve 10) to a native heart valve, including the native aortic valve of a patient. One or more of these functions may include steerability of the delivery catheter 330 (for example via catheter deflection); fine axial positioning adjustment of the prosthetic heart valve; positioning of the prosthetic heart valve rotationally around its longitudinal axis for commissure alignment; and/or motorized, automatic, or semi-automatic balloon inflation and deflation for easy and accurate deployment of the prosthetic heart valve.

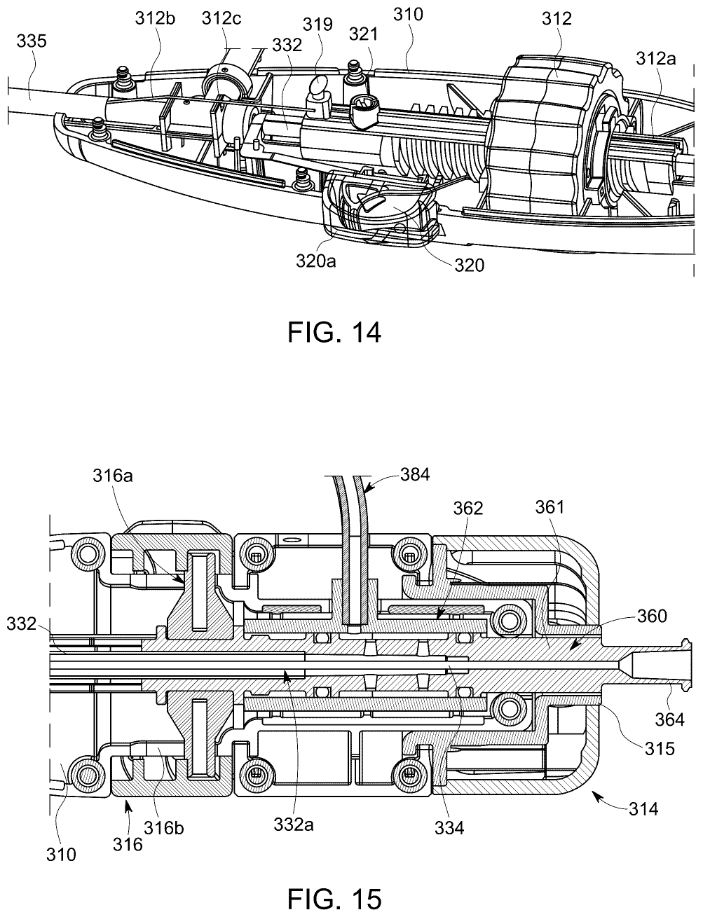

The steerability of delivery system 300 is described in greater detail below in connection with FIGS. 12-14. However, it should be understood that components and functionality of the steerability of delivery system 100 and delivery system 300 may be interchangeable. The primary mechanism of achieving steerability of delivery catheter is a steering actuator, which in the illustrated embodiment has the form of a rotatable steering knob 312, which in some examples may include ridges and protrusions to enhance grip of the steering knob 312 by a user. The steering knob 312 may be received within the handle 310 so that the steering knob 312 is rotatable relative to a longitudinal axis of the handle 310 but fixed axially relative to the handle 310. Referring now to FIGS. 12-14, with particular emphasis on FIG. 14, a lead screw or carriage 312a may be positioned within the handle 310 and may include exterior threads that are configured to engage or intermesh with the internal threads of steering knob 312. Preferably, the carriage 312a is rotationally fixed (about its longitudinal axis) relative to the handle 310. Although there may be many ways to achieve this, in the illustrated example, a bottom end of the carriage 312a (in a position generally opposite the position of the steering wire 312b and indicator 319) includes a recess or groove running in a longitudinal or axial direction, and the housing includes a flange, wall, or rail that runs in the longitudinal or axial direction and which is received within the groove of the carriage 312a. With this configuration, the carriage 312a may slide axially along the rail in the handle, but the carriage 312a is not able to rotate about its central longitudinal axis. It should be understood that, in FIG. 14, only a portion of the handle 310 is shown. For example, the handle 310 may be provided with a shell, where the shell is split into two pieces (e.g. a bottom half shown in FIG. 14 and a top half omitted form FIG. 14), where the two pieces snap together or otherwise couple together to form the handle 310 shown in FIGS. 12-13. However, it should be understood that the handle 310 may include other types of casings or shells, including one-piece or three-or-more piece shells, without deviating from the concepts described herein.

Referring still to FIG. 14, as a user rotates the steering knob 312, the steering knob 312 rotates about its central axis without moving axially. As the interior threads of the steering knob 312 intermesh with the exterior threads of the carriage 312a. Because the carriage 312a is unable to rotated about its longitudinal axis, rotation of the steering knob 312 forces the carriage 312a to translate either distally or proximally within the handle 310 depending on the direction that the steering knob 312 is rotated. A proximal end portion of the steering wire 312b may be fixed to the carriage 312a, for example a distal end portion of the carriage 312a. Thus, as the steering knob 312 is rotated to draw the carriage 312a proximally, it tends to pull the steering wire 312b proximally to translate and/or tension the steering wire 312b, whereas when the steering knob 312 is rotated to draw the carriage 312a distally, it tends to push the steering wire 312b distally to translate and/or relax the steering wire 312b. One or more stops may be provided on the carriage 312a (and/or within the handle 310) to provide proximal and distal limits of translation of the carriage 312a within the handle. Preferably, when the carriage 312a is at its distalmost position relative to the handle 310, similar to the position shown in FIGS. 12 and 14, the steering wire 312b has little or no tension, and the delivery catheter 330 has little or no deflection due to the steering wire 312b.

Still referring to FIGS. 12-14, the steering wire 312b may extend distally from the carriage 312a all the way to a distal end portion of the delivery catheter 330. In one example, a steering ring is fixed to a distal end portion of the delivery catheter 330, for example to a steering ring fixed to the steering catheter 335 near the proximal end of the balloon (e.g. balloon 136 or a similar balloon). In embodiments in which a distal end portion of the steering wire 312b is coupled to a steering ring fixed to the steering catheter 335, the steering wire 312b may extend through a steering wire lumen formed in the wall of the steering catheter 335. However, in other embodiments, the steering wire 312b may extend inside or outside of the steering catheter 335. Still further, in other embodiments the steering wire 312b may be coupled to a distal end portion of the outer catheter 332 or the inner catheter 334, or the nosecone (e.g. nosecone 138). Regardless of the particular construction, to perform a steering operation, the user may rotate steering knob 312 to translate the carriage 312a proximally, which in turn translates the steering wire 312b proximally and/or tensions the steering wire 312b. The tension on the steering wire 312b is applied to the delivery catheter 330 where the steering wire 312b connects to the delivery catheter 330, causing the delivery catheter 330, and particularly a distal end portion or tip portion thereof, to deflect to attempt to resolve the tension. An example of this deflection is shown in FIG. 13. As the carriage 312a translates proximally, and indicator 319 which is attached to the carriage 312a and which protrudes through, and is visible in, a window 318 of the housing 310, also translates proximally. The indicator 319 is best shown in FIG. 14, where it is in a distal-most position indicating little or no deflection. However, as the steering (or deflection) operation progresses, the indicator 319 moves farther proximally within window 318 to provide an indicator of the amount of deflection achieved. Compare, for example, the position of indicator 319 in FIG. 12, which indicates little or no deflection of the delivery catheter 330, to the position of indicator 319 in FIG. 13 which indicates a large degree of deflection of the delivery catheter 330. The range of length that the indicator 319 may move, which may correspond about 1:1 to actual catheter deflection, may be between about 1.0 inches (about 2.54 cm) and about 1.6 inches (about 4.064 cm), including about 1.3 inches (about 3.302 cm). As described above in connection with delivery system 100, the steering operation may be used to help better guide the delivery catheter 330, particularly a distal end portion thereof, along tortuous vessel anatomy, such as tracking around the aortic arch during an aortic valve replacement procedure, as well as axially and radially centering the prosthetic heart valve 10 within the native valve annulus.

Still referring to FIG. 14, in some examples, the handle 310 includes one or more steering wire guides 312c to assist with maintaining a desired position of the steering wire 312b inside the handle 310. For example, in the illustrated embodiment, the handle 310 includes at least one steering wire guide 312c, which may take the form of a plate with a notch that receives the steering wire 312b. However, more supports or different types of supports or guides may be provided, or the steering wire guides or supports may be omitted altogether.

Delivery system 300, similar to delivery system 100, may include a mechanism to rotate the prosthetic heart valve on the delivery catheter 330 about its central longitudinal axis to achieve commissure alignment, as well as a mechanism to finely adjust the axial position of the prosthetic heart valve on the delivery catheter 330 without needing to move the entire handle 310 axially. Although in some examples one or both of these features may be omitted, in the illustrated example, both features are included and provide functionality without hindering each other.

Referring again briefly to FIGS. 12 and 13, the handle 310 may include a commissure alignment actuator 314 and an axial alignment actuator 316. These actuators may have similar or identical functionality to the corresponding actuators of delivery system 100, and similar or identical positioning, although in these illustrated embodiments, the commissure alignment actuator 314 is positioned at or near the terminal proximal end of the handle 310. Also, similar to delivery system 100, the commissure alignment actuator 314 and axial alignment actuator 316 may each take any suitable form, although in the illustrated example, each actuator is provided as a rotatable wheel or knob.

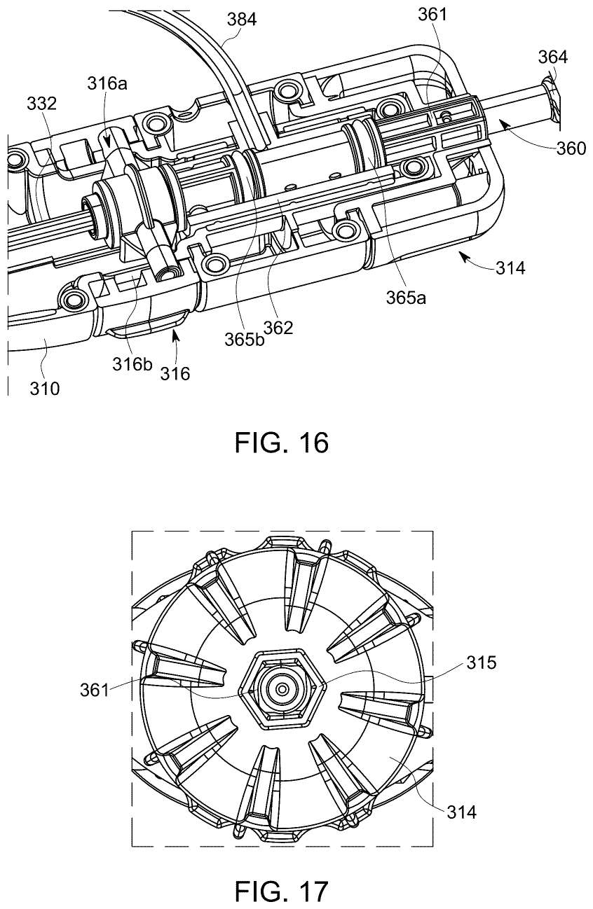

FIG. 15 is a cross-section of a proximal end of the handle 310, while FIG. 16 shows a similar perspective view of interior components of the handle 310 with certain components shown fully. As explained elsewhere herein, to achieve commissure alignment, the delivery catheter 330, and in particular in this example the outer catheter 332, is rotated about its longitudinal axis, which causes rotation of the balloon (e.g. balloon 136) and the prosthetic heart valve (e.g. prosthetic heart valve 10) mounted thereon. Similarly, to achieve fine axial depth adjustment, the delivery catheter 330, and in particular in this example the outer catheter 332, is advanced or retracted axially relative to the handle 310. Because both operations act on the outer catheter 332, the two mechanisms for commissure alignment and fine axial depth adjustment are preferably decoupled from each other so that each operation can be achieved independently of each other.

Referring to FIGS. 15-16, the delivery system 300 may be provided with a catheter hub 360 at least partially within handle 310, the catheter hub 360 being fixed to the outer catheter 332 so that rotation of the catheter hub 360 causes corresponding rotation of outer catheter 332, and so that axial movement of the catheter hub 360 causes corresponding axial movement of outer catheter 332. The catheter hub 360 may be generally cylindrical and hollow, with a proximal end of the outer catheter 332 received within and connected to a distal end portion of the catheter hub 360. In the illustrated embodiment, near or at a distal end of the catheter hub 360, two flanges (e.g. circular flanges) may be positioned at a spaced distance from each other, with an axial adjustment carrier 316a coupled to the catheter hub 360 between the two flanges. The axial adjustment carrier 316a may have a cylindrical recess which can be snap fit or otherwise coupled to the catheter hub 360 between the two flanges, such that the axial adjustment carrier 316 is rotatable relative to the catheter hub 360, but axially fixed relative to the catheter hub 360. The axial adjustment carrier 316a may include extensions that pass through corresponding slots 316b in the handle 310. These extensions may engage internal threads of the axial alignment actuator 316. With this configuration, as the axial alignment actuator 316 is rotated, the axial adjustment carrier 316a is forced to advance distally or retract proximally as the interaction between the extensions and the slots 316b prevent the axial adjustment carrier 316a from rotation about its central longitudinal axis relative to the handle 310. As the axial adjustment carrier 316a advances distally or retracts proximally, the interaction between the axial adjustment carrier 316a and the two flanges of the catheter hub 360 causes corresponding axial advancement or retraction of the catheter hub 360, and in turn corresponding axial advancement or retraction of the outer catheter 332.

Referring now to FIGS. 15-17, a proximal portion of the catheter hub may include a keyed area 361 that is received within a corresponding keyed area 315 of the commissure alignment actuator 314. In the illustrated example, the keyed area 361 has a hexagonal shape, while the keyed area 315 has a corresponding hexagonally-shaped recess. With this configuration, rotation of the commissure alignment actuator 314 (and thus the keyed area 315) causes corresponding rotation of the keyed area 361 (and thus the catheter hub 360. Although the corresponding shape is described and shown as hexagonal, it should be understood that any other shape that permits transmission of torque may be suitable, including square-shaped, triangular-shaped, “plus-sign” shaped, etc. It should be understood that the fit of the keyed area 361 within keyed area 315 may be loose (or otherwise not significantly tight) so that the catheter hub 360 is capable of moving axially relative to the commissure alignment actuator 314 despite the keyed fit. The keyed area 361 may have a length that is large enough so that, as the catheter hub 360 moves through its maximum range of axial motion, the keyed area 361 always maintains at least some contact with the keyed area 315. As a user rotates the commissure alignment wheel 314, and corresponding rotation of the balloon catheter 360 (and outer catheter 332) results, it should be understood that the axial alignment carrier 316a does not rotate, since the catheter hub 360 is freely (or substantially freely) rotatable relative to the axial alignment carrier 316a. With the above-described configuration, axial movement of the catheter hub 360 (and thus the outer catheter 332) via actuation of the axial alignment actuator 316 is independent from rotational movement of the catheter hub 360 (and thus the outer catheter 332) via actuation of the commissure alignment actuator 314. It should be understood that, in the illustrated embodiment, a guidewire port 364 is provided at the proximal end of the catheter hub 360 to assist with passing a guidewire (such as guidewire GW) through the inner catheter 334 (also referred to as an internal guidewire lumen) extending through the handle 310 and the delivery catheter 330.

Although a configuration is described above that allows for independent rotation and axial depth adjustment of the outer catheter 332 via two independent actuators 314, 316 on the handle 310, it is also important to maintain the ability to ensure a fluid-tight connection between the handle 310 and the balloon (e.g. balloon 116) at the distal end of the delivery catheter 330 to allow for inflation of the balloon. It is also important that the fluid line (e.g. tubing 384 described below) remains stationary despite the rotation and/or translation of the outer catheter 332. By remaining stationary, the fluid line is prevented from getting tangled with or otherwise interfering with other components of the delivery system 300, such as a guidewire or any cables (e.g. data transmission cables) coupled to the handle 310. Referring to FIGS. 15, 16, and 18, one mechanism for achieving the desired fluid connection is shown. In the illustrated example, a hub housing 362 is provided around a portion of the catheter hub 360. In the illustrated embodiment, the hub housing 362 is generally cylindrical and hollow and receives the catheter hub 360 therethrough, with the hub housing 362 being rotationally and axially fixed relative to the handle 310. As shown in FIGS. 15, 16 and 18, tubing 384 may pass into the handle 310 and connect to a port in the hub housing 362. An opposite end of the tubing 384 (not shown) may be connected to an inflation system such as inflation system 170, for example to a distal end of a syringe (see FIGS. 8-10 for example). Two seals 365a, 365b may be positioned between the catheter hub 360 and the hub housing 362. As shown in FIGS. 15, 16, and 18, the seals 365a, 365b may take the form of o-rings, gaskets, or other seals that are positioned on opposite sides of the inflation port of the hub housing 362. The spacing is preferably such that, through the entire range of available axial motion of the catheter hub 360 relative to the hub housing 362, the seals 365a, 365b remain on opposite sides of the inflation port of the hub housing 362. It should also be understood that the catheter hub 360 is capable of rotating about its longitudinal axis relative to the hub housing 362. A void space may be formed between the two seals 365a, 365b, the interior surface of the hub housing 362, and the exterior surface of the catheter hub 360, with the void space configured to receive fluid or other inflation media, such as saline, therein. The catheter hub 360 may include one, two, or more openings in a wall thereof so that fluid may pass from the void space to the interior of the catheter hub 360, without leaking between the catheter hub 360 and the hub housing 362 due to the seals 365a, 365b.

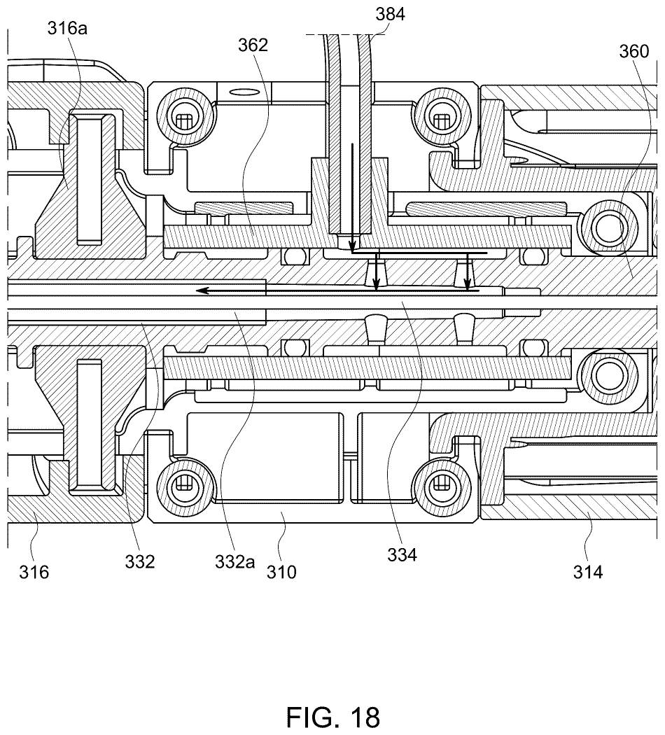

As best shown in FIGS. 15 and 18, as inflation media is advanced from an inflation system through tubing 384, and through the inflation port of hub housing 362, the inflation media enters the interior of catheter hub 360. When in use, a guidewire (e.g. guidewire GW) extends through the inner catheter 334 of the catheter hub 360. The inner catheter 334 may extend through an interior channel of the catheter hub 360, and the inner catheter 334 may plug a proximal end area of the catheter hub 360 so that inflation media cannot flow proximally to exit the proximal end of the catheter hub 360 via guidewire port 364. Instead, the inflation media must flow (when positive pressure is applied) through an inflation lumen 332a between the interior of the outer catheter 332 and the exterior of the inner catheter 334. This flow path is illustrated in FIG. 18 with arrows. The inflation lumen 332a leads, at its distal end, to the interior of the balloon (e.g. balloon 116) so that the balloon may be inflated, and the prosthetic heart valve (e.g. prosthetic heart valve 10) crimped thereon may be expanded and deployed into the native valve annulus.

Although in some embodiments, inflation media may be passed manually into (or withdrawn manually from) the inflation lumen 332a, in the illustrated embodiment, motorized or semi-automatic inflation is provided. The inflation system used with the delivery system 300 may be similar or identical to inflation system 170. Referring to FIG. 14, a balloon actuator 320 may be provided on the handle, which may be similar or identical to balloon actuator 120. In some embodiments, a cap or protector 320a may be provided so that, when the cap 320a is covering the balloon actuator 320, the balloon actuator is unlikely or impossible to be unintentionally actuated. The cap 320a may be removably coupled to the handle 310, hingedly coupled to the handle 310, or configured in any other way so that the cap 320a may be adjusted or removed so that a user can access the balloon actuator 320 at the desired time. Other features, such as a pin that must be pulled from the balloon actuator 320 (similar to the pin of fire extinguisher or grenade) may be provided through balloon actuator 320 to prevent unintentional activation. The balloon actuator 320 may be operably coupled to the inflation system, for example via a flex circuit such as that shown in FIG. 14, via wires (similar or identical to those shown in FIGS. 8-10), and/or via a combination of rigid and flexible circuits that connected to the electronic switch so that, upon actuation of balloon actuator 320, inflation media is either passed from the inflation system toward the balloon to inflate the balloon, or withdrawn from the balloon toward the inflation to deflate the balloon, in a similar or identical manner to as described above in connection with delivery system 100.

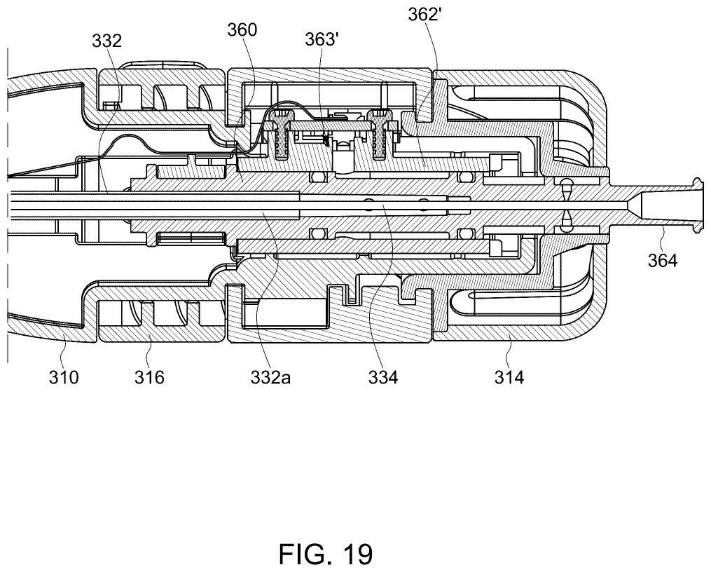

FIG. 19 shows a cross-section of a proximal end of the handle 310 with an alternate hub housing 362′ Hub housing 362′ may be similar to hub housing 362, with the main difference being that a pressure sensor 363′ is mounted to the hub housing 362′ so that the pressure sensor 363′ is in fluid communication with the pathway of the inflation media (for example the pressure sensor 363′ may face inwardly toward the hub housing 362′ at or near the inflation port. The pressure sensor 363′ may be operably coupled (e.g. via the wired connection shown in FIG. 19 or via any other suitable connection, including wireless connections) to the inflation system (e.g. inflation system 170) or to a computer operably associated with the inflation system. With this configuration, a stable pressure tap is provided for pressure monitoring within the hub housing 362′ to provide real time pressure feedback to the user and/or to the inflation system, which feedback may in turn be used by the user and/or the inflation system to adjust the amount and/or rate of inflation or deflation of the balloon. In some embodiments, the pressure sensor 363′ is capable of detecting a range of pressures between about −1 atm and about 12 atm. It should be understood that the pressure sensor may be provided in different locations, and similar pressure sensors may be provided with any of the other embodiments described herein.

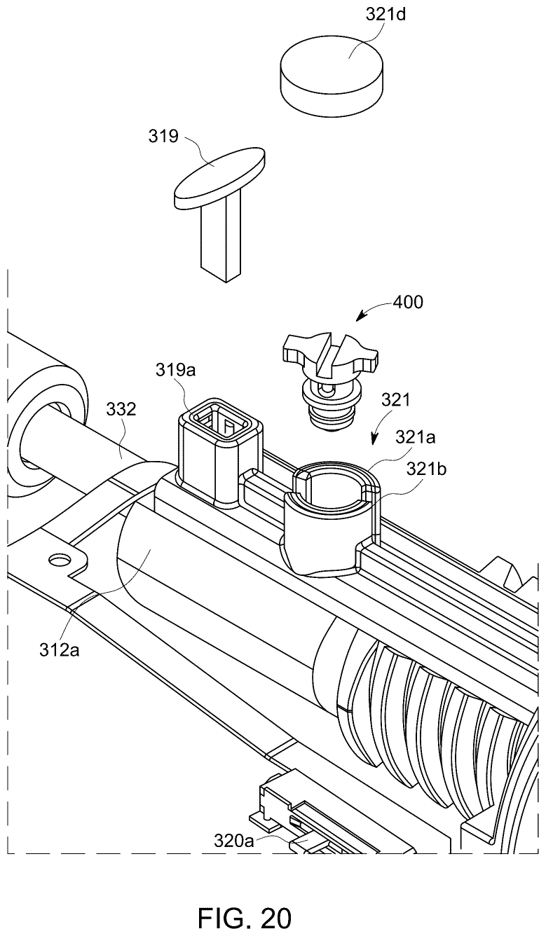

Referring briefly back to FIG. 14, it is described above that the proximal end of the steering wire 312b may be coupled to the carriage 312a. In one particular example, the proximal end of the steering wire 312b may be coupled to the carriage 312a via being coupled to a receiver 321 that is coupled to (e.g. formed integrally with) the carriage 312a. FIG. 20 shows an exploded view of a portion of the handle 310. In FIG. 20, the indicator 319 is shown spaced away from an indicator hub 319a that receives the indicator 319 therein. Similarly, in FIG. 20, a 400 steering wire coupler 400 is shown spaced away from the receiver 321 that receives the steering wire coupler 400 therein.

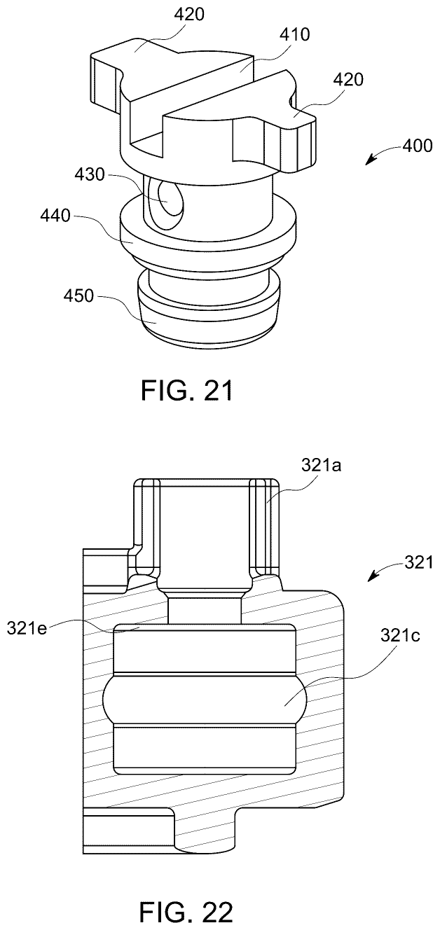

FIG. 21 shows an enlarged perspective view of the steering wire coupler 400. As shown in FIG. 21, the coupler 400 may include a top or proximal end that is generally cylindrical, but for a channel 410 extending through the top end (the channel 410 being open at the top of the channel), and two tabs 420 extending radially outwardly form the top end of the coupler 400. In the illustrated example, a line passing through the center of the channel 410 is substantially perpendicular to a line passing through the centers of the two tabs 420. A through-hole 430 may extend through a shaft portion of the coupler 400. The shaft containing the through-hole 430 may have a smaller diameter than both the top portion of the coupler 400 and a middle flange 440 positioned below the through-hole 430. As explained in greater detail below, the steering wire 312b may be passed fully through the through-hole 430, and then partially spooled around the shaft, which may form a groove between the larger diameter top end and middle flange 440. The bottom end of the coupler 400 may include another flange 450 that forms a local large diameter section to help secure the coupler 400 to the receiver 321, as described in greater detail below. In some examples, the coupler 400 may be formed of a hard or rigid material, such as a metal or hard polymer. In one example, the coupler 400 may be formed of stainless steel, such as 304 stainless steel.

Referring back to FIG. 20, during assembly, the proximal end of the steering wire 312b may be threaded through the through-hole 430 of the coupler 400, and the coupler 400 may be rotated to spool the steering wire 312b around the shaft of the coupler 400 between the top end of the coupler and the middle flange 440 of the coupler. In one example, the coupler 400 may be rotated between about 180 degree and about 360 degrees, including about 270 degrees, to force the steering wire 312b to spool around the coupler 400. In some examples, the channel 410 may be configured to receive a tool, such as a screwdriver, to assist with such rotation. After performing this rotation and spooling, the tabs 420 of the coupler may be aligned with two corresponding axial slots 321b of the receiver 321. As shown in FIG. 20, the receiver may include two arcuate upward extensions 321a that together form two axial slots 321b, the two axial slots 321b being positioned along a line. In some embodiments, including that shown in FIG. 20, the line passing through the two axial slots 321b may be angularly offset from a central longitudinal axis of the carriage 312a. With this configuration, the imaginary line passing through the two axial slots 321b may avoid passing through the indicator hub 319a, which may provide better clearance for routing the steering wire 312b. Once the steering wire 312b has been inserter through the through-hole 430 of the coupler 400 and the coupler 400 has been rotated, while the tabs 420 are aligned with the axial slots 321b, the coupler 400 may be inserted downwardly or pushed into the receiver 321. FIG. 22 is a cross-section of a portion of the receiver 321. As shown in FIG. 22, the receiver 312c may include an opening 321c, which may be configured to receive the outer catheter 332 therethrough. A shoulder 321e may separate the opening 321c from the upward extensions 321a, such that a relatively narrow opening separates the larger opening 321c from upward extensions 321a. When the coupler 400 is pushed fully into the receiver 321, the shaft between the middle flange 440 and the bottom flange 450 may pass through the narrow opening, such that the upper surface of the bottom flange 450 engages the bottom surface of the shoulder 321e. The interaction of the bottom flange 450 within the shoulder 321e may help ensure the coupler 400 cannot be easily pulled upwardly out of the receiver 321 after the coupler 400 is pushed into the receiver 321.

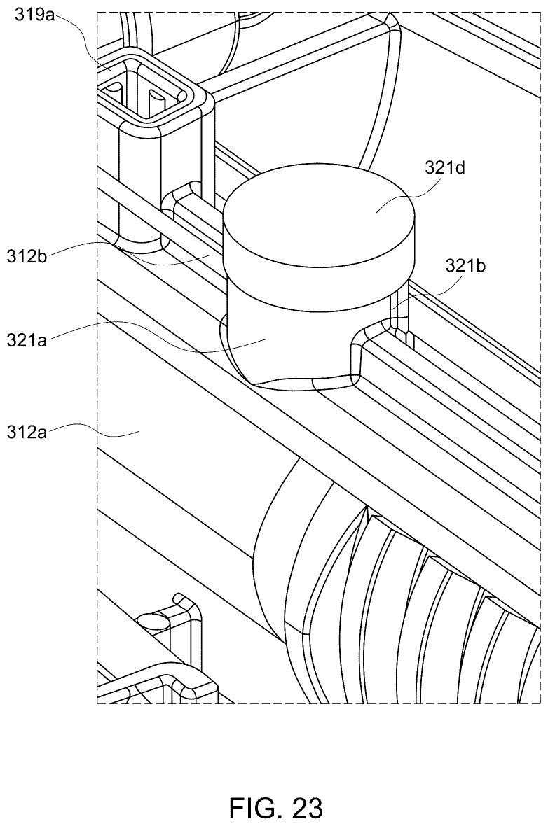

The function of the steering wire 312b is described in greater detail above. However, it should be noted that while the steering wire 312b is tensioned, the steering wire 312b will tend to try to rotate the coupler 400 to unspool the steering wire 312b from the coupler 400. However, the tabs 420 being received within the axial slots 321b result in contact between the tabs 420 and the upward extensions 321a, with such contact generally preventing any rotation of the coupler. In some examples, the receiver 321 is formed of a thermoplastic such as acetal (including the material offered under the tradename Delrin offered by Delrin USA, LLC). Depending in part on the material used to form the upward extensions 321a of the receiver, and depending in part on the thickness of the walls forming the upward extensions 321a, at high enough tensile forces on the steering wire 312b, the torque applied by the coupler 400 onto the receiver 321 may cause the upward extensions 321a to splay outwardly, which could result in the coupler 400 being allowed to rotate and the steering wire 321b to unwind from the coupler 321. One way to prevent this result, as shown in FIG. 23, is by providing a receiver cap 321d that is placed over the receiver 321. The exterior surface of the top portions of the upward extensions 321a may be in contact with an inner surface of the cap 321d, such that the cap 321d provide support to help the upward extensions 321a resist the tendency to splay outwardly under high forces. It should be understood that, if cap 321d is provided, it is assembled to the receiver 321 after the steering wire 312b is coupled to, and partially spooled around, the coupler 400, and after the coupler 400 has been inserted into the receiver 321. In some examples, the cap 321d is formed of a rigid material, such as a hard polymer or metal, including for example stainless steel (e.g. 304 stainless steel). However, in other examples, the cap 321d may be omitted. If the cap 321d is omitted, and if it is desired to provide additional support to the upward extensions 321a to prevent splaying, the upward extensions 321a may be formed with a greater thickness and/or of a material that has high strength or rigidity.

Still referring to FIG. 23, after assembly of the steering wire 312b to the coupler 400, and the coupler 400 to the receiver 321 (whether or not a receiver cap 321d is used), the steering wire 312b may exit the receiver through the axial slot 321b closer to the indicator hub 319a. As shown in FIG. 23, the steering wire 312b may exit the receiver with a trajectory that allows the steering wire 312b to avoid contacting the indicator hub 319a. In some examples, the steering wire 312b may be formed as a flat wire (e.g. a wire having a substantially rectangular transverse cross-section). In one example in which the steering wire 312b is a flat wire, it may be a 0.006 inch (0.1524 mm) by 0.020 inch (0.508 mm) flat wire. However, the steering wire 312b may be a flat wire with different dimensions, or a round wire instead of a flat wire.

Delivery system 300 may be used in generally the same manner as described in connection with delivery system 100 (including as shown in FIG. 11, for example) and thus is not described in further detail herein. Delivery system 300, like delivery system 100, allows for catheter deflection, active commissure alignment, and active depth alignment with a single user-friendly system. An electronic switch may also be provided to communicate with the inflation system to precisely control valve deployment and implantation. All of these features may be useable, in an intuitive way, with single-operator control. In other words, a user may relatively easily be able to manipulate the handle 310 (or handle 110) with one or two hands, being able to use all functions, including inflation, steering, active commissure alignment, and active depth alignment, without needing a second operator to manipulate any of these controls.

It should be understood that features of delivery system 300 not explicitly described in connection with FIGS. 12-19 may be similar or identical to components shown and/or described in connection with delivery system 100, or may have any other suitable structure and/or function.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A prosthetic heart valve delivery system comprising:
a handle;
a delivery catheter extending distally from the handle;
an inflatable balloon positioned at a distal end portion of the delivery catheter;
a prosthetic heart valve configured to be collapsed onto the inflatable balloon for transcatheter delivery of the prosthetic heart valve to a native heart valve of a patient;
an axial adjustment actuator positioned on the handle, wherein actuation of the axial adjustment actuator causes the prosthetic heart valve to translate distally or proximally relative to the handle when the prosthetic heart valve is collapsed onto the inflatable balloon; and
a commissure alignment actuator positioned on the handle, wherein actuation of the commissure alignment actuator causes the prosthetic heart valve to rotate about a central longitudinal axis of the prosthetic heart valve when the prosthetic heart valve is collapsed onto the inflatable balloon;
wherein the axial adjustment actuator and the commissure alignment actuator are configured to be independently actuated so that actuation of the axial adjustment actuator does not cause the prosthetic heart valve to rotate about the central longitudinal axis of the prosthetic heart valve, and actuation of the commissure alignment actuator does not cause the prosthetic heart valve to translate distally or proximally relative to the handle.

2. The prosthetic heart valve delivery system of claim 1, wherein the delivery catheter includes an outer catheter, and an inner catheter radially inward of the outer catheter.

3. The prosthetic heart valve delivery system of claim 2, further comprising a catheter hub positioned at least partially within the handle, the catheter hub being axially and rotatably fixed to the inner catheter.

4. The prosthetic heart valve delivery system of claim 3, further comprising an axial adjustment carrier mounted to the catheter hub so that the axial adjustment carrier is axially fixed relative to the catheter hub but is rotatable about the catheter hub.

5. The prosthetic heart valve delivery system of claim 4, wherein the axial adjustment carrier includes an extension, and the axial adjustment actuator includes internal threading, the extension engaging the internal threading.

6. The prosthetic heart valve delivery system of claim 5, wherein the extension extends through a slot formed in the handle, the slot defining a maximum length of translation of the axial adjustment carrier relative to the handle.

7. The prosthetic heart valve delivery system of claim 4, wherein the catheter hub includes a keyed portion received within a keyed recess of the commissure alignment actuator so that rotation of the commissure alignment actuator causes corresponding rotation of the catheter hub.

8. The prosthetic heart valve delivery system of claim 7, wherein the keyed portion is hexagonal and the keyed recess is hexagonal.

9. The prosthetic heart valve delivery system of claim 7, wherein the keyed portion is positioned at a proximal end portion of the catheter hub, and the keyed portion has a length, the length being selected so that the keyed portion remains engaged with the keyed recess in all achievable axial positions of the catheter hub relative to the handle.

10. The prosthetic heart valve delivery system of claim 7, further comprising a hub housing at least partially surrounding the catheter hub, the hub housing including an inflation port configured to receive a first end of an inflation tubing.

11. The prosthetic heart valve delivery system of claim 10, wherein the hub housing is axially and rotationally fixed relative to the housing.

12. The prosthetic heart valve delivery system of claim 10, further comprising a first seal engaged to an outer surface of the catheter hub at a first location, and a second seal engaged to the outer surface of the catheter hub at a second location, the first seal and the second seal also engaged to an inner surface of the hub housing.

13. The prosthetic heart valve delivery system of claim 12, wherein the catheter hub includes at least one aperture in a wall of the catheter hub at a location between the first seal and the second seal to create a first fluid pathway from (a) the inflation port into a void space defined between (i) the inner surface of the hub housing, (ii) the outer surface of the catheter hub, (iii) the first seal, and (iv) the second seal, and (b) from the void space into an interior lumen of the catheter hub.

14. The prosthetic heart valve delivery system of claim 13, wherein the inner catheter is fixed to create a second fluid pathway from the interior lumen of the catheter hub, through the inner catheter, and to the balloon, the first fluid pathway being continuous with the second fluid pathway.

15. The prosthetic heart valve delivery system of claim 14, further comprising a pressure sensor in contact with the first fluid pathway and/or the second fluid pathway.

16. The prosthetic heart valve delivery system of claim 12, wherein the catheter hub is rotatable and translatable relative to the hub housing.

17. The prosthetic heart valve delivery system of claim 16, wherein the catheter hub has a total range of translation relative to the hub housing, and at all points within the total range of translation, the inflation port is positioned between the first seal and the second seal.

18. The prosthetic heart valve delivery system of claim 7, further comprising a steering actuator coupled to the handle, and a carriage within the handle engaged with the steering actuator so that actuation of the steering actuator causes distal or proximal translation of the carriage relative to the handle.

19. The prosthetic heart valve delivery system of claim 18, further comprising a steering wire having a proximal end portion coupled to the carriage, and a distal end portion coupled to a steering ring on a distal end portion of the delivery catheter, so that distal or proximal translation of the carriage causes deflection of the distal end portion of the delivery catheter.

20. The prosthetic heart valve delivery system of claim 19, further comprising a deflection indicator slot in the handle, a deflection indicator coupled to the carriage and extending through the deflection indicator slot so that a proximal-to-distal position of the deflection indicator relative to the deflection indicator slot indicates an amount of deflection of the distal end portion of the delivery catheter.
